(19) United States
(12) Patent Application Publication
(10) Pub. No.: US 2020/0187777 A1
(43) Pub. Date: Jun. 18, 2020

(54) Digital Therapeutic Component to Optimize Induction of Buprenorphine-Containing Products

(71) Applicant: Pear Therapeutics, Inc., Boston, MA (US)

(72) Inventors: Hilary Luderer, Boston, MA (US); Nicole Enman, Boston, MA (US); Audrey Kern, Boston, MA (US); Jake Nichols, Boston, MA (US); Yuri Maricich, Boston, MA (US)

(21) Appl. No.: 16/715,680

(22) Filed: Dec. 16, 2019

Related U.S. Application Data
(60) Provisional application No. 62/779,705, filed on Dec. 14, 2018.

(51) Int. Cl. A61B 5/00; A61B 5/024; G16H 80/00; G16H 20/10; G16H 10/60

(52) U.S. Cl. CPC A61B 5/0022; A61B 5/4836; A61B 5/02444; G16H 80/00; A61K 31/485; G16H 10/60; A61B 2562/0219; A61B 2562/0257; G16H 20/10

(57) ABSTRACT

A system comprising data processing hardware and memory hardware in communication with the data processing hardware, the memory hardware storing instructions that when executed on the data processing hardware cause the data processing hardware to perform operations comprising executing a prescription digital therapeutic configured to treat symptoms associated with opioid use disorder in a patient, wherein executing the prescription digital therapeutic comprises receiving a plurality of inputs associated with the patient from one or more of (i) first sensors associated directly with the patient and (ii) second sensors associated with a patient electronic device, wherein the plurality of inputs represent a level of opioid withdrawal associated with the patient, weighting the plurality of inputs associated with the patient to provide a plurality of weighted inputs, determining a recommended dosage of a buprenorphine-containing product for the patient based on the plurality of weighted inputs, and instructing an administration unit to administer the recommended dosage of the buprenorphine-containing product to the patient.

### CROSS REFERENCE TO RELATED APPLICATIONS

This U.S. patent application claims priority under 35 U.S.C. § 119(e) to U.S. Provisional Application 62/779,705, filed on Dec. 14, 2018. The disclosure of this prior application is considered part of the disclosure of this application and is hereby incorporated by reference in its entirety.

### FIELD

The present disclosure relates to digital therapeutics and, more particularly, to systems and methods for improving adherence to and induction of drug therapies, such as buprenorphine-containing products, using digital therapeutics.

### BACKGROUND

The information provided in this section is for the purpose of generally presenting the context of the disclosure. Work of the presently named inventors, to the extent it is described in this section, as well as aspects of the description that may not otherwise qualify as prior art at the time of filing, are neither expressly nor impliedly admitted as prior art against the present disclosure.

Opioid Use Disorder (OUD) is a major public health problem that affected more than 2.2 million people in the US in 2018. OUD is a chronic disease that creates a significant burden on the healthcare system; the misuse of opiate analgesics is estimated to cost insurers $72.5 billion each year. In 2017, 47,600 overdose deaths were attributed to opioids and 15,482 deaths were related to heroin, resulting in a daily average of approximately 130 lethal opioid overdoses per day in 2017.

Medication-Assisted Treatment (MAT) may provide therapy with FDA-approved medications, such as buprenorphine-containing products for OUD. MAT, or any other combination of therapy and medication, may be applied to any suitable disease or disorder, including, but not limited to, substance abuse disorder, insomnia, depression, schizophrenia, traumatic brain injury, epilepsy, post-traumatic stress disorder (PTSD), Parkinson's disease, multiple sclerosis, autism spectrum disorder, migraines, etc. MAT may similarly combine therapy (e.g., cognitive behavioral therapy or “CBT”) with any suitable medication(s) for these diseases and disorders. Such medications may include conventional pharmaceuticals (e.g., small-molecule drugs, which are usually derived from chemical synthesis) and/or biopharmaceuticals (e.g., recombinant proteins, vaccines, blood products used therapeutically for gene therapy, monoclonal antibodies, cell therapy, etc.).

While MAT for OUD has proven effective and creates a high medical value, there are a number of limitations associated with the standard of care that result in high unmet medical needs. An overwhelming majority of individuals who need treatment (80-90%) do not receive care. This is most often due to refusal to seek treatment, high cost of care, stigma associated with care, and/or lack of or limited access to treatment. For those who do seek OUD treatment, lack of medication adherence, and high attrition rates limit effectiveness. There are many reasons why patients discontinue buprenorphine pharmacotherapy, including unpleasant experiences during induction, such as unalleviated withdrawal symptoms and cravings, a perception that “the medication doesn't work,” barriers such as medication cost and difficulty keeping medical appointments, the desire to continue using illicit opioids, and patients' perceptions of low risk for relapse when discontinuing treatment. Induction is a particularly critical period, as patients who have negative experiences may not continue treatment beyond the first day of dosing.

Accordingly, systems and methods for improving adherence to and induction of drug therapies, such as drug therapies utilizing buprenorphine-containing products, may be desired.

### SUMMARY

One aspect of the disclosure provides a system including data processing hardware and memory hardware in communication with the data processing hardware, the memory hardware storing instructions that when executed on the data processing hardware cause the data processing hardware to perform operations comprising executing a prescription digital therapeutic configured to treat symptoms associated with opioid use disorder in a patient. In one example, executing the prescription digital therapeutic comprises receiving a plurality of inputs associated with the patient from one or more of (i) first sensors associated directly with the patient and (ii) second sensors associated with a patient electronic device, wherein the plurality of inputs represent a level of opioid withdrawal associated with the patient. The operations comprise weighting the plurality of inputs associated with the patient to provide a plurality of weighted inputs, determining a recommended dosage of a buprenorphine-containing product for the patient based on the plurality of weighted inputs, and instructing an administration unit to administer the recommended dosage of the buprenorphine-containing product to the patient.

Implementations of the disclosure may include one or more of the following features. In some implementations, the system may include the administration unit, wherein the administration unit is configured to administer the recommended dosage of the buprenorphine-containing product to the patient based on the instructions. The operations may include administering the recommended dosage of the buprenorphine-containing product to the patient.

The prescription digital therapeutic may be configured to implement cognitive behavioral therapy to treat the symptoms associated with the opioid use disorder. The first sensors associated directly with the patient may include one or more of: (i) a heart rate monitor, (ii) a blood pressure monitor, (iii) a sleep monitor, (iv) an electrodermal activity monitor, (v) a skin temperature sensor, and (vi) a sweat monitor. The second sensors associated with the patient electronic device may include one or more of: (i) an accelerometer, (ii) a proximity sensor, and (iii) an activity monitor.

In some implementations, weighting the plurality of inputs associated with the patient may include assigning a first weight to a first input of the plurality of inputs and a second weight different than the first weight to a second input of the plurality of inputs. The first input of the plurality of inputs may include the first sensors associated directly with the patient and the second input of the plurality of inputs may include the second sensors associated with the patient electronic device. The first weight may be greater than the second weight. The administration unit may include one or more of: (i) a delivery pump, (ii) an injection unit, (iii) an implant, (iv) an oral absorption unit, (v) an inhaler, and (vi) a nasal injector.

Another aspect of the disclosure provides a method comprising executing a prescription digital therapeutic configured to treat symptoms associated with opioid use disorder in a patient. In one example, executing the prescription digital therapeutic comprises receiving, by data processing hardware, a plurality of inputs associated with the patient from one or more of (i) first sensors associated directly with the patient and (ii) second sensors associated with a patient electronic device, wherein the plurality of inputs represent a level of opioid withdrawal associated with the patient, weighting, by the data processing hardware, the plurality of inputs associated with the patient to provide a plurality of weighted inputs, determining, by the data processing hardware, a recommended dosage of a buprenorphine-containing product for the patient based on the plurality of weighted inputs, and instructing, by the data processing hardware, an administration unit to administer the recommended dosage of the buprenorphine-containing product to the patient.

Implementations of the disclosure may include one or more of the following features. In some implementations, the administration unit may be configured to administer the recommended dosage of the buprenorphine-containing product to the patient based on the instructions. Executing the prescription digital therapeutic may include administering, by the administration unit, the recommended dosage of the buprenorphine-containing product to the patient.

The prescription digital therapeutic may be configured to implement cognitive behavioral therapy to treat the symptoms associated with the opioid use disorder. The first sensors associated directly with the patient may include one or more of: (i) a heart rate monitor, (ii) a blood pressure monitor, (iii) a sleep monitor, (iv) an electrodermal activity monitor, (v) a skin temperature sensor, and (vi) a sweat monitor. The second sensors associated with the patient electronic device may include one or more of: (i) an accelerometer, (ii) a proximity sensor, and (iii) an activity monitor.

In some implementations, weighting the plurality of inputs associated with the patient may include assigning a first weight to a first input of the plurality of inputs and a second weight different than the first weight to a second input of the plurality of inputs. The first input of the plurality of inputs may include the first sensors associated directly with the patient and the second input of the plurality of inputs may include the second sensors associated with the patient electronic device. The first weight may be greater than the second weight. The administration unit may include one or more of: (i) a delivery pump, (ii) an injection unit, (iii) an implant, (iv) an oral absorption unit, (v) an inhaler, and (vi) a nasal injector.

Another aspect of the disclosure provides a system for executing a prescription digital therapeutic configured to treat symptoms associated with opioid use disorder in a patient, the system comprising: an input module configured to receive a plurality of inputs associated with the patient from one or more of (i) first sensors associated directly with the patient and (ii) second sensors associated with a patient electronic device, wherein the plurality of inputs represent a level of opioid withdrawal associated with the patient, an input weighting module configured to weight the plurality of inputs associated with the patient to provide a plurality of weighted inputs, a recommendation module configured to determine a recommended dosage of a buprenorphine-containing product for the patient based on the plurality of weighted inputs, and an administration module configured to instruct an administration unit to administer the recommended dosage of the buprenorphine-containing product to the patient.

The foregoing modules may be implemented by any suitable data processing hardware (e.g., one or more processors) executing instructions stored in suitable memory hardware in accordance with the structures and techniques set forth herein.

The details of one or more implementations of the disclosure are set forth in the accompanying drawings and the description below. Other aspects, features, and advantages will be apparent from the description, drawings, and claims.

### DESCRIPTION OF DRAWINGS

FIG. 1 is a schematic view of a system for displaying and managing patient data including a digital therapeutic in accordance with an exemplary embodiment of the present disclosure;

FIG. 2 is a schematic view of a system for optimizing induction of a prescription drug using the digital therapeutic of FIG. 1;

FIG. 3 is a schematic flowchart representing an exemplary determination of a dosage of a prescription drug on a first day;

FIG. 4 is a schematic flowchart representing an exemplary determination of a dosage of a prescription drug on a second day;

FIG. 5 is a schematic flowchart representing an exemplary determination of a dosage of a prescription drug on a third day and later;

FIG. 6 is a schematic flowchart representing exemplary startup operations of a patient application associated with the digital therapeutic of FIG. 1;

FIG. 7 is a flowchart illustrating a method for implementing a prescription digital therapeutic configured to treat symptoms associated with opioid use disorder in a patient, in accordance with an exemplary embodiment of the present disclosure; and

FIG. 8 is a schematic view of an example computing device that may be used to implement the systems and methods described herein.

Like reference symbols in the various drawings indicate like elements.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

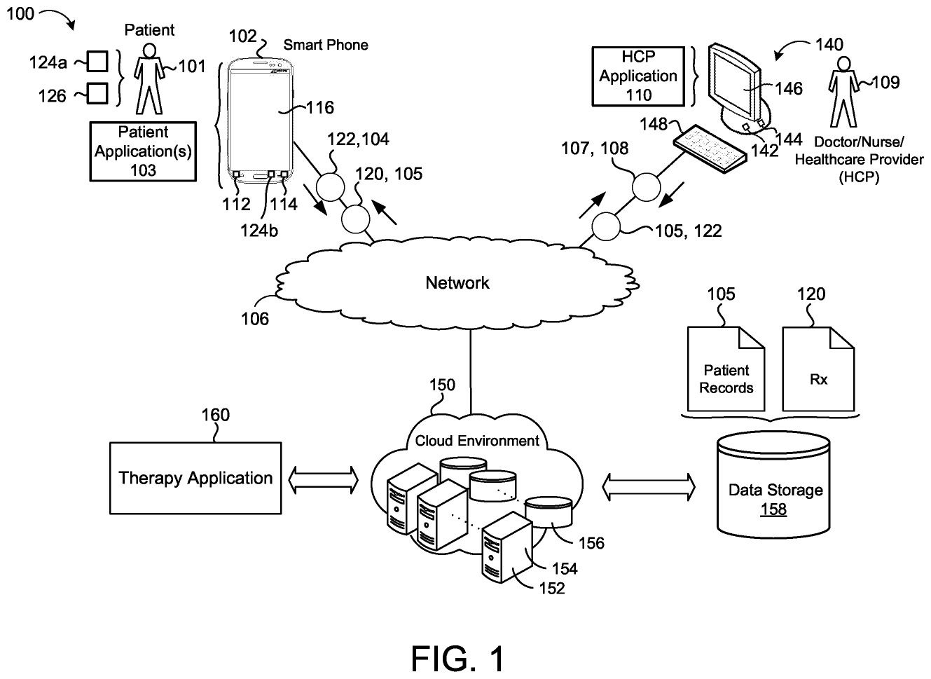

Referring to FIG. 1, in some implementations, a therapy prescription system 100 provides a patient 101 access to a prescription digital therapeutic 120 prescribed to the patient 101 and monitors events associated with the patient's 101 interaction with the prescription digital therapeutic 120. Although the digital therapeutic 120 is described herein as being a “prescription” digital therapeutic, it is understood that, according to some implementations, the digital therapeutic 120 will not require a prescription from a clinician. Rather, in such implementations, the digital therapeutic 120 may be available to a patient without a prescription, and the digital therapeutic 120 nonetheless otherwise functions in accordance with the description of the prescription digital therapeutic 120 described herein. According to implementations in which the digital therapeutic 120 is not prescribed, the person using or being administered the digital therapeutic may be referred to as a “user.” A “user” may include a patient 101 or any other person using or being administered the digital therapeutic 120, irrespective of whether the digital therapeutic 120 was prescribed to that person.

As used herein, a digital therapy may also be referred to as a digital-therapeutic configured to deliver evidence-based psychosocial intervention techniques for treating a patient with a particular disease or disorder, as well as symptoms and/or behaviors associated with the particular disease or disorder. As one example, the patient 101 may be diagnosed with a chronic disease and the prescription digital therapeutic 120 may be specifically tailored for addressing one or more symptoms associated with the chronic disease that the patient 101 may experience. In some implementations, the digital therapeutic 120 may include or be combined with traditional drug therapy (e.g., buprenorphine-containing products, such as stand-alone buprenorphine, products including buprenorphine and other drugs such as naloxone, etc.), which similarly may or may not require a prescription. An authorized healthcare provider (HCP) 109 (e.g., a doctor, nurse, etc.) may prescribe the patient 101 the prescription digital therapeutic 120 designed to treat symptoms in the patient 101. The HCP 109 may include a physician, nurse, clinician, or other qualified health professionals. The HCP 109 may provide any suitable level of supervision to the patient 101, including little to no supervision.

In some examples, the system 100 includes a network 106, a patient device 102, an HCP system 140, and a therapy application 160. The network 106 provides access to cloud computing resources 150 (e.g., distributed system) that execute the therapy application 160 to provide for the performance of services on remote devices. Accordingly, the network 106 allows for interaction between patients 101 and HCPs 109 with the therapy application 160. For instance, the therapy application 160 may provide the patient 101 access to the prescription digital therapeutic 120 and receive user input or event data 122 inputted by the patient 101 associated with the patient's 101 interaction with the prescription digital therapeutic 120. In turn, the therapy application 160 may store the event data 122 on a storage resource 156.

The network 106 may include any type of network that allows sending and receiving communication signals, such as a wireless telecommunication network, a cellular telephone network, a time division multiple access (TDMA) network, a code division multiple access (CDMA) network, Global system for mobile communications (GSM), a third generation (3G) network, fourth generation (4G) network, fifth generation (5G) network, a satellite communications network, and other communication networks. The network 106 may include one or more of a Wide Area Network (WAN), a Local Area Network (LAN), and a Personal Area Network (PAN). In some examples, the network 106 includes a combination of data networks, telecommunication networks, and a combination of data and telecommunication networks. The patient device 102, the HCP system 140, and the therapy application 160 communicate with each other by sending and receiving signals (wired or wireless) via the network 106, which, in some examples, may utilize Bluetooth, Wi-Fi, etc. In some examples, the network 106 provides access to cloud computing resources, which may be elastic/on-demand computing and/or storage resources 156 available over the network 106. The term “cloud” services generally refers to a service delivered from one or more remote devices accessible via one or more networks 106, rather than a service performed locally on a user's device.

The patient device 102 may include, but is not limited to, a portable electronic device (e.g., smartphone, cellular phone, personal digital assistant, laptop computer, or wireless tablet device), a desktop computer, or any other electronic device capable of sending and receiving information via the network 106. The patient device 102 includes data processing hardware 112 (a computing device that executes instructions), memory hardware 114, and a display 116 in communication with the data processing hardware 112. In some implementations, the patient 101 may be connected to first sensor(s) 124a, such as a heart rate sensor or monitor, blood pressure sensor or monitor, a sleep sensor, an activity monitor (e.g., an electrodermal activity monitor), a skin temperature sensor, a sweat monitor, and/or any other suitable sensors or monitors (e.g., wearable sensors or monitors) in communication with the patient device 102. Additionally, or alternatively, the patient device may include second sensor(s) 124b, such as an accelerometer, proximity sensor, an activity or exercise monitor, etc., to provide data about the patient 101 and/or the patient's 101 interaction with the patient device 102. In some implementations, the patient 101 and the patient device 102 may be in communication with an administration unit 126, such as a delivery pump, an injection unit, an implant, an oral absorption unit (e.g., a sublingually dissolvable film or pill), an inhaler, a nasal injector, other transmucosal administration units, etc. For example, the patient device 102 may transmit a recommendation and/or an instruction to the administration unit 126 for a dosage of medication the patient 101 should take. In some examples, the patient device 102 includes a keyboard, mouse, microphones, and/or a camera for allowing the patient 101 to input data. In addition to or in lieu of the display 116, the patient device 102 may include one or more speakers to output audio data to the patient 101. For instance, audible alerts may be output by the speaker to notify the patient 101 about some time sensitive event associated with the prescription digital therapeutic 120.

In some implementations, the patient device 102 executes the patient application 103 (or accesses a web-based patient application) for establishing a connection with the therapy application 160 to access the prescription digital therapeutic 120. For instance, the patient 101 may have access to the patient application 103 for a duration (e.g., 3 months) of the prescription digital therapeutic 120 prescribed to the patient 101. Here, the patient device 102 may launch the patient application 103 by initially providing an access code 104 when the prescription digital therapeutic 120 is prescribed by the HCP 109, the access code 104 allowing the patient 101 to access content associated with the prescription digital therapeutic 120 from the therapy application 160. The content may be specifically tailored for treating/addressing one or more symptoms associated with the specific indication that the patient 101 may be experiencing. The patient application 103, when executing on the data processing hardware 112 of the patient device 102, is configured to display a variety of graphical user interfaces (GUIs) on the display 116 of the patient device 102 that, among other things, allow the patient 101 to (i) input event data 122 describing one or more parameters associated with the patient 101 (e.g., an indication of how the patient 101 is feeling, an indication of the last time the patient 101 used a drug, an indication of where the patient 101 was located when they last used a drug, an indication of who the patient 101 was with last time they used a drug, an indication of the time of day the patient 101 last used a drug, etc.); (ii) solicit information from the patient 101; (iii) deliver therapeutic content (e.g., CBT content) to the patient 101; (iv) allow the patient 101 to contact their HCP 109; (v) allow the patient 101 to review their progress adhering to their prescription regimen with respect to the prescription digital therapeutic 120 and/or any prescribed medication; and/or (vi) present journal entries for the patient 101 to view and/or edit.

The storage resources 156 may provide data storage 158 for storing the event data 122 received from the patient 101 in a corresponding patient record 105 as well as the prescription digital therapeutic 120 prescribed to the patient 101. The patient record 105 may be encrypted while stored on the data storage 158 so that any information identifying patient 101 is anonymized, but may later be decrypted when the patient 101 or supervising HCP 109 requests the patient record 105 (assuming the requester is authorized/authenticated to access the patient record 105). All data transmitted over the network 106 between the patient device 102 and the cloud computing system 150 may be encrypted and sent over secure communication channels. For instance, the patient application 103 may encrypt the event data 122 before transmitting to the therapy application 160 via the HTTPS protocol and decrypt a patient record 105 received from the therapy application 160. When network connectivity is not available, the patient application 103 may store the event data 122 in an encrypted queue within the memory hardware 114 until network connectivity is available.

The HCP system 140 may be located at a clinic, doctor's office, or facility administered by the HCP 109 and includes data processing hardware 142, memory hardware 144, and a display 146. The memory hardware 144 and the display 146 are in communication with the data processing hardware 142. For instance, the data processing hardware 142 may reside on a desktop computer or portable electronic device for allowing the HCP 109 to input and retrieve data to and from the therapy application 160. In some examples, the HCP 109 may initially onboard some or all of patient data 107 at the time of prescribing the prescription digital therapeutic 120 to the patient 101. The HCP system 140 includes a keyboard 148, mouse, microphones, speakers and/or a camera.

In some implementations, the HCP system 140 (i.e., via the data processing hardware 142) executes the HCP application 110 (or accesses a web-based patient application) for establishing a connection with the therapy application 160 to input and retrieve data therefrom. For instance, the HCP system 140 may be able to access the anonymized patient record 105 securely stored by the therapy application 160 on the storage resources 156 by providing an authentication token 108 validating that the HCP 109 is supervising the patient 101 and authorized to access the corresponding patient record 105. The authentication token 108 may identify the particular patient 101 associated with the patient record 105 that the HCP system 140 is permitted to obtain from the therapy application 160. The patient record 105 may include time-stamped event data 122 indicating the patient's interaction with the prescription digital therapeutic 120 through the patient application 103 executing on the patient device 102. The HCP application 110, when executing on the data processing hardware 142 of the HCP system 140, is configured to display a variety of graphical user interfaces (GUIs) on the display 146 of the HCP system 140 that, among other things, allow the HCP 109 to input event data 122 describing one or more parameters associated with the patient 101, solicit information from the patient 101, and input clinical notes associated with the patient 101.

In some implementations, the HCP application 110 is in communication with a single patient application 103 for a single patient 101 and manages data associated with the single patient application 103. In other implementations, the HCP application 110 is in communication with several patient applications 103 associated with several patients 101, and the HCP application 110 may manage and display the data associated with the several patient applications 103 in any suitable manner, e.g., by toggling between different views and/or displaying certain data simultaneously. In other implementations, the HCP application 110 is in communication with multiple patient applications 103 for the same patient 101 and simultaneously manages data associated with the multiple patient applications 103. In this implementation, the data from multiple patient applications 103 may be displayed simultaneously in any suitable manner or the data from each patient application 103 may be displayed discretely such that the HCP 109 is able to toggle between the discretely displayed data.

The cloud computing resources 150 may be a distributed system (e.g., remote environment) having scalable/elastic resources 152. The resources 152 include computing resources 154 (e.g., data processing hardware) and/or the storage resources 156 (e.g., memory hardware). The cloud computing resources 150 execute the therapy application 160 for facilitating communications with the patient device 102 and the HCP system 140 and storing data on the storage resources 156 within the data storage 158. In some examples, the therapy application 160 and the data storage 158 reside on a standalone computing device. The therapy application 160 may provide the patient 101 with the patient application 103 (e.g., a mobile application, a web-site application, or a downloadable program that includes a set of instructions) executable on the data processing hardware 112 and accessible through the network 106 via the patient device 102 when the patient 101 provides a valid access code 104. Similarly, the therapy application 160 may provide the HCP 109 with the HCP application 110 (e.g., a mobile application, a web-site application, or a downloadable program that includes a set of instructions) executable on the data processing hardware 142 and accessible through the network 106 via the HCP system 140.

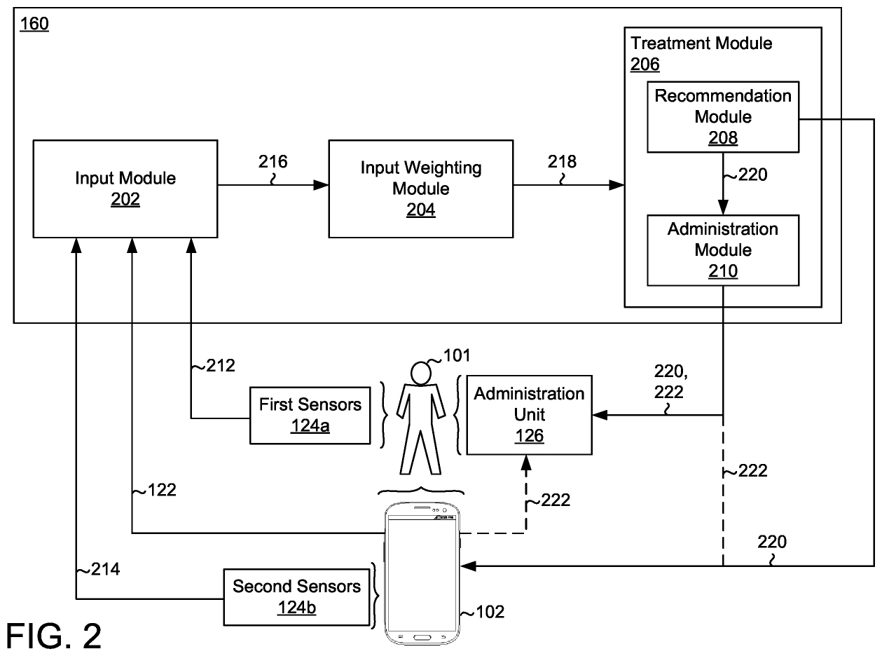

Referring to FIG. 2, the therapy application 160 may include an input module 202, an input weighting module 204, and a treatment module 206 having a recommendation module 208 and an administration module 210. It should be understood that fewer or greater modules may be implemented, and certain modules may be combined or separated as suitable. As set forth above, the therapy application 160 may be executed by the cloud computing resources 150 and may be in communication with the patient device 102 associated with the patient 101 via the network 106.

The therapy application 160 is configured to facilitate remote inductions by patients 101, i.e., inductions that are performed outside of the observation of the HCP 109 (e.g., within the patient's home). That is, the system 100 described herein facilitates remote supervision of the patient 101 by the HCP 109, without the need for the HCP 109 to witness and monitor the patient 101 taking the medication in-person. In some implementations, the HCP 109 may prescribe the patient 101 with the therapy application 160 combined with a prescription drug designed for remote inductions, after the HCP 109 meets with the patient 101 and determines that the patient 101 meets the criteria for the therapy application 160 and remote inductions. According to this exemplary implementation, the HCP 109 may provide explicit instructions to the patient 101 regarding how and when the patient 101 should start taking the medication, along with clear requirements for maintaining contact with the HCP 109 via the therapy application 160, telephone contact, messaging contact, etc. As another example, the therapy application 160 may provide instructions to the patient 101 regarding how and when the patient 101 should take the medication. For example, in one implementation, the therapy application 160 may provide instructions to the patient 101 regarding how and when the patient 101 should take the medication based on at least historical data associated with the patient's 101 prior interaction with the therapy application 160 and/or historical data associated with other patients' prior interaction with the therapy application 160.

The input module 202 is configured to obtain (e.g., fetch or receive) one or more inputs including event data 122 from the storage resource 156 of the patient device 102, first sensor data 212 from the first sensor(s) 124a associated with or connected to the patient 101, second sensor data 214 from the second sensor(s) 124b associated with the patient device 102, etc. In some examples, the input module 202 may actively monitor and gather data from the patient device 102 and/or the sensors 124a, 124b. In other examples, the input module 202 may query the patient 101 to provide information or data from the patient device 102 and/or sensors 124a, 124b. The input module 202 is configured to transmit data to, and be in communication with, the input weighting module 204.

The input weighting module 204 is configured to receive input data 216 from the input module 202. Based on the input data 216, the input weighting module 204 is configured to assign a weight or value to each of the one or more inputs to generate weighted input data 218. For example, the first sensor(s) 124a may include a heart rate monitor and the first sensor data 212 may include heart rate data associated with the patient 101. In such an example, the input weighting module 204 may assign a different (e.g., greater or lesser) value to the heart rate data than, for example, event data 122 inputted by the patient 101. The input weighting module 204 is configured to be in communication with and transmit the weighted input data 218 to the treatment module 206.

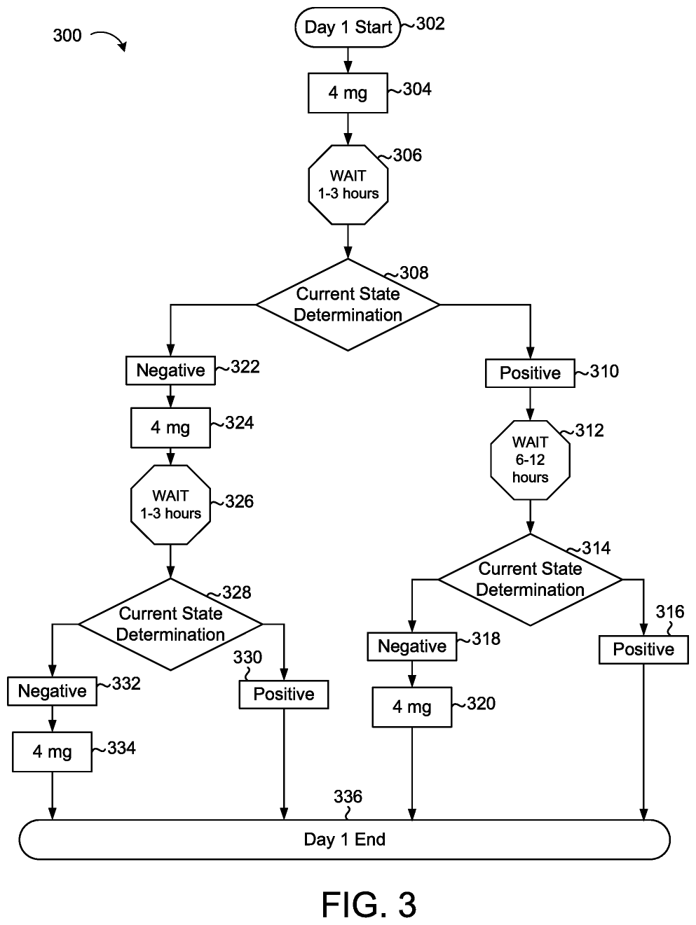

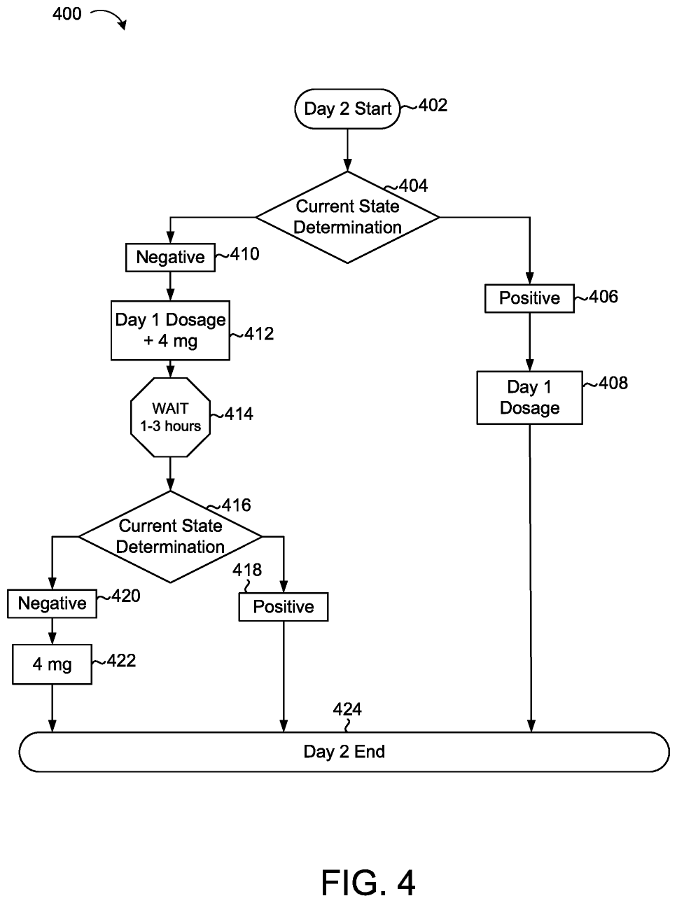

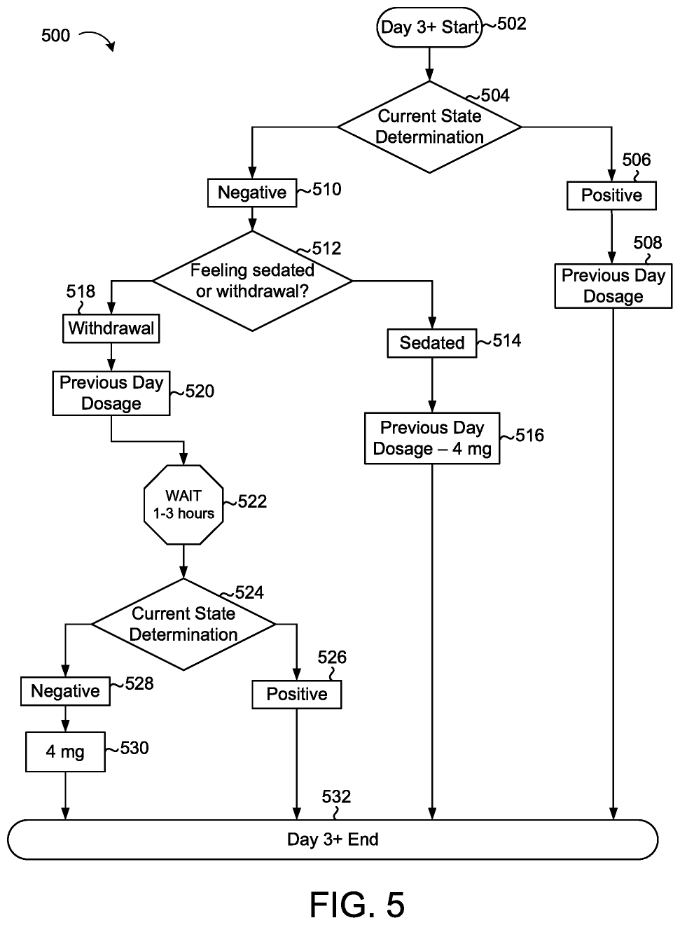

The treatment module 206 is configured to receive the weighted input data 218 from the input weighting module 204. The recommendation module 208 of the treatment module 206 is configured to determine a recommended dosage for the patient 101 based on the weighted input data 218 according to one or more processes further described herein with respect to FIGS. 3-6. In the examples described herein, the recommended dosage pertains to one or more dosages of buprenorphine-containing products designed to treat OUD, however, it should be understood that the recommended dosage may pertain to any suitable medication for any suitable disease or disorder. While the recommended dosage and the algorithm for determining the recommended dosage are designed to be suitable for a patient 101 taking a medication remote from the HCP 109 (e.g., at the patient's home), the recommended dosage and algorithm may similarly be implemented for a patient 101 taking a medication under direct supervision of an HCP 109 or in any other suitable situation.

The recommendation module 208 is configured to transmit recommended dosage data 220 associated with the recommended dosage to the administration module 210. The recommendation module 208 is also configured to transmit, via the network 106, the recommended dosage data 220 to the patient device 102, causing the patient device 102 to display the recommended dosage on the display 116 in the patient application 103. In some implementations, the recommendation module 208 is configured to automatically transmit the recommended dosage data 220 to the patient device 102 at predetermined intervals (e.g., every 8 hours), or the recommendation module 208 is configured to transmit the recommended dosage data 220 in response to event data 122 inputted by the patient 101 on the patient device 102.

In some implementations, the patient 101 may be associated with the administration unit 126, which, as set forth above, may include, for example, a delivery pump, an injection unit, an implant, an oral absorption unit (e.g., a sublingually dissolvable film or pill), an inhaler, a nasal injector, other transmucosal administration units, etc. The administration unit 126 may be in communication with the administration module 210. The administration module 210 is configured to transmit dosage instruction data 222 and/or the recommended dosage data 220 to the administration unit 126. In some implementations, the administration module 210 is configured to transmit the dosage instruction data 222 to the administration unit 126, which causes the administration unit 126 to administer or deliver the instructed dosage to the patient 101. In such implementations, the administration unit 126 may automatically administer or deliver the recommended dosage to the patient 101, or the administration unit 126 may query the patient 101 and/or the HCP 109 to confirm the recommended dosage before administering the recommended dosage to the patient 101. In some implementations, the administration module 210 may be in communication with and configured to transmit the dosage instruction data 222 to the patient device 102 and the patient device 102 may be in communication with and configured to transmit the dosage instruction data 222 to the administration unit 126.

Referring to FIGS. 3-7, flowcharts representing methods 300, 400, 500, 600, and 700 for determining the recommended dosage on specific days are generally shown. The methods 300, 400, 500, 600, 700 may be performed by the therapy application 160 (e.g., the input weighting module 204 and the treatment module 206), along with any other components of the therapy prescription system 100. The algorithms 300, 400, 500, 600, 700 illustrated in FIGS. 3-7 may be performed on the back end by the therapy application 160, and the patient 101 may be shown any suitable display on the display 116 of the patient device 102. The methods 300, 400, 500, 600, 700 may include interaction between the patient 101 and the patient application 103 on the patient device 102 and/or the HCP 109. In some implementations, the patient 101 may access the patient application 103 on their own accord. Additionally, or alternatively, the patient application 103 is configured to prompt the patient 101 to access the patient application 103, e.g., via a notification such as a visual alert (e.g., via the display 116) and/or an audial alert (e.g., via speakers of the patient device 102).

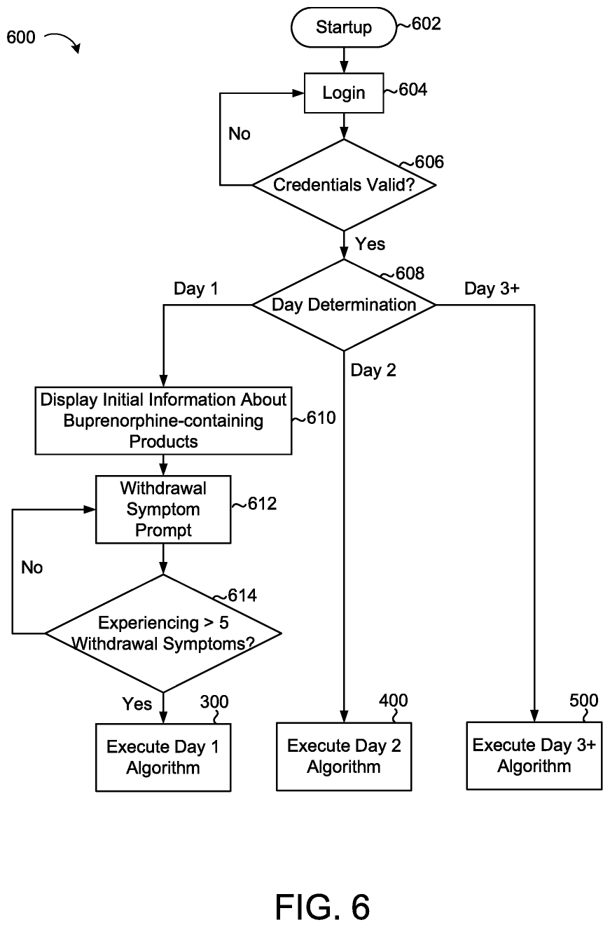

Referring to FIGS. 3 and 6, the patient 101 may access the therapy application 160 through the patient application 103 on the first day (Day 1) that the patient 101 is prescribed the therapy application 160 and buprenorphine-containing products. The patient application 103 is configured to display on the display 116 initial general information about buprenorphine-containing products including how buprenorphine-containing products work, types of buprenorphine-containing products, risks associated with buprenorphine-containing products, side effects of buprenorphine-containing products, etc. On Day 1, the patient application 103 is configured to query the patient 101 (e.g., via the patient device 102) to determine whether the patient 101 has used any opioids recently (e.g., within the last 12-72 hours), so as to verify that all opioid drugs are out of the patient's 101 system. The patient application 103 may display a plurality of withdrawal symptoms, including, but not limited to: (i) joint aches, (ii) bone aches, (iii) chills, (iv) sweating, (v) anxiety, (vi) irritability, (vii) twitching, (viii) tremors, (ix) shaking, (x) goosebumps, (xi) restlessness, (xii) heavy yawning, (xiii) enlarged pupils, (xiv) runny eyes, (xv) cramps, (xvi) nausea, (xvii) vomiting, and/or (xviii) diarrhea, etc. The patient application 103 is configured to query the patient 101 (e.g., via the patient device 102) to determine whether the patient 101 is experiencing, for example, at least five of the above symptoms associated with withdrawal, any cravings for opioids, etc. In some implementations, the patient 101 may not be allowed to proceed if the patient 101 is not experiencing, for example, at least five withdrawal symptoms and/or any cravings for opioids.

Upon determination that the patient 101 is experiencing withdrawal, the patient application 103 is configured to query the patient 101 (e.g., via the patient device 102) to determine a particular dosage or dosage range of the buprenorphine-containing product that the patient 101 has been prescribed and possesses. For example, buprenorphine-containing products may be formed as a film strip or a pill in a variety of dosages (e.g., 2 mg, 4 mg, 8 mg). In some implementations, if the patient application 103 determines that the patient 101 has an incorrect or unsuitable dosage of the buprenorphine-containing product, the patient application 103 is configured to provide instructions (e.g., via the patient device 102), such as an instructional video, step-by-step instructions, etc., for how to cut or otherwise modify the buprenorphine-containing product into the correct dosage for the patient 101.

On Day 1 start 302, the patient application 103 is configured to instruct the patient to take the initial recommended dosage at 304 (e.g., 4 mg), or a specific dosage which may be pre-determined by the HCP 109 of the patient 101 or determined in any suitable manner, e.g., setting 4 mg as the default initial recommended dosage for all patients 101. It should be understood that 4 mg is used as an exemplary dosage for the methods 300, 400, 500, and that any suitable dosage may be implemented based on at least the type of buprenorphine-containing product (e.g., brand name, potency, etc.), the duration of effects of the buprenorphine-containing product (e.g., short-acting, long-acting, etc.), and/or the specific label of the buprenorphine-containing product. The patient application 103 is then configured to instruct the patient 101 to wait 1-3 hours at 306. After waiting, the patient application 103 is configured to determine a current state of the patient 101 at 308. The patient 101 may input or report their current state (e.g., event data 122) by selecting a positive indicator at 310 or a negative indicator at 322, for example, that are displayed on the display 116. The positive indicator may include a smiley face, a thumbs up, a check mark, text, or any other suitable graphical representation. The negative indicator may include a frowny face, a thumbs down, a cross, text, or any other suitable graphical representation. Additionally, or alternatively, the patient 101 may indicate their current state by speaking into the microphone of the patient device 102, by gesturing to the patient device 102, or in any other suitable manner. In some implementations, the patient application 103 may supplement or replace the event data 122 inputted by the patient 101 with any other suitable data, including the first sensor data 212, the second sensor data 214, etc.

If the patient application 103 determines, at 308, that the current state of the patient 101 is positive, e.g., based on the patient 101 selecting the positive indicator at 310, then the patient application 103 is configured to instruct the patient 101 to wait 6-12 hours at 312. After waiting, the patient application 103 is configured to determine a current state of the patient 101 at 314. If the patient application 103 determines, at 314, that the current state of the patient 101 is positive, e.g., based on the patient 101 selecting the positive indicator at 316, then no action is needed, and the Day 1 algorithm 300 ends at 336. If the patient application 103 determines, at 314, that the current state of the patient 101 is negative, e.g., based on the patient 101 selecting the negative indicator at 318, then the patient application 103 is configured to instruct the patient 101 to take 4 mg of the buprenorphine-containing product at 320 and the Day 1 algorithm 300 ends at 336.

After waiting the initial 1-3 hours at 306, if the patient application 103 determines, at 308, that the current state of the patient 101 is negative, e.g., based on the patient 101 selecting the negative indicator at 322, then the patient application 103 is configured to instruct the patient 101 to take 4 mg of the buprenorphine-containing product at 324 and wait 1-3 hours at 326. After waiting, the patient application 103 is configured to determine a current state of the patient 101 at 328. If the patient application 103 determines, at 328, that the current state of the patient 101 is positive, e.g., based on the patient 101 selecting the positive indicator at 330, then no action is needed, and the Day 1 algorithm 300 ends at 336. If the patient application 103 determines, at 328, that the current state of the patient 101 is negative, e.g., based on the patient 101 selecting the negative indicator at 332, then the patient application 103 is configured to instruct the patient 101 to take 4 mg of the buprenorphine-containing product at 334 and the Day 1 algorithm 300 ends at 336.

The Day 2 algorithm 400 may be dependent upon the total dosage determined from the Day 1 algorithm 300. Day 2 start 402 may be when the patient 101 wakes up or at any suitable time during the second day. At Day 2 start 402, the patient application 103 is configured to query the patient 101, at 404, regarding a current state of the patient 101. The patient application 103 may be configured to query the patient 101, at 404, regarding their current state of withdrawal, such as that set forth above with respect to the Day 1 method 300 (e.g., determining if the patient 101 is experiencing at least five withdrawal symptoms and/or any cravings for opioids). If the patient application 103 determines, at 404, that the current state of the patient 101 is positive, e.g., based on the patient 101 selecting the positive indicator at 406, then the patient application 103 is configured to instruct the patient to take the Day 1 dosage at 408 and the Day 2 algorithm 400 ends at 424. If the patient application 103 determines, at 404, that the current state of the patient 101 is negative, e.g., based on the patient 101 selecting the negative indicator at 410, then the patient application 103 is configured to instruct the patient to take the Day 1 dosage plus 4 mg at 412 and to wait 1-3 hours at 414. After waiting, the patient application 103 is configured to determine a current state of the patient 101 at 416. If the patient application 103 determines, at 416, that the current state of the patient 101 is positive, e.g., based on the patient 101 selecting the positive indicator at 418, then no action is needed, and the Day 2 algorithm 400 ends at 424. If the patient application 103 determines, at 416, that the current state of the patient 101 is negative, e.g., based on the patient 101 selecting the negative indicator at 420, then the patient application 103 is configured to instruct the patient 101 to take 4 mg of the buprenorphine-containing product at 422 and the Day 2 algorithm 400 ends at 424.

The Day 3+ algorithm 500 for the third day and later of the patient's 101 program may be dependent upon the previous day dosage. For example, the Day 3 dosage is dependent upon the Day 2 dosage, the Day 4 dosage is dependent upon the Day 3 dosage, and so on. Day 3+ start 502 may be when the patient 101 wakes up or at any suitable time during the third or later day. At Day 3+ start 502, the patient application 103 is configured to query the patient 101, at 504, regarding a current state of the patient 101. If the patient application 103 determines, at 504, that the current state of the patient 101 is positive, e.g., based on the patient 101 selecting the positive indicator at 506, then the patient application 103 is configured to instruct the patient to take the previous day dosage at 508 and the Day 3+ algorithm 500 ends for that day at 532. If the patient application 103 determines, at 504, that the current state of the patient 101 is negative, e.g., based on the patient 101 selecting the negative indicator at 510, then the patient application 103 is configured to query the patient 101, at 512, regarding whether the patient 101 is feeling sedated (e.g., overly tired or overly sleepy) or experiencing withdrawal. If the patient application 103 determines, at 512, that the patient 101 is feeling sedated, e.g., based on the patient 101 selecting a sedation indicator at 514, then the patient application 103 is configured to instruct the patient to take the previous day dosage minus 4 mg at 516 and the Day 3+ algorithm 500 ends for that day at 532. If the patient application 103 determines, at 512, that the patient 101 is feeling withdrawal, e.g., based on the patient 101 selecting a withdrawal indicator at 518, then the patient application 103 is configured to instruct the patient 101 to take the previous day dosage at 520. To determine, at 512, whether the patient 101 is feeling sedated or withdrawal, the patient application 103 may supplement or replace the event data 122 inputted by the patient 101 with any other suitable data, including the first sensor data 212, the second sensor data 214, etc. After the patient 101 takes the previous day dosage at 518, then the patient application 103 is configured to instruct the patient 101 to wait 1-3 hours at 522. After waiting, the patient application 103 is configured to determine a current state of the patient 101 at 524. If the patient application 103 determines, at 524, that the current state of the patient 101 is positive, e.g., based on the patient 101 selecting the positive indicator at 526, then no action is needed, and the Day 3+ algorithm 500 ends for that day at 532. If the patient application 103 determines, at 524, that the current state of the patient 101 is negative, e.g., based on the patient 101 selecting the negative indicator at 528, then the patient application 103 is configured to instruct the patient 101 to take 4 mg of the buprenorphine-containing product at 530 and the Day 3+ algorithm 500 ends for that day at 532.

By Day 4 and beyond, the daily dosage amount for the patient 101 is designed to be adequately determined, but the Day 3+ method 500 allows for some variation based on the state of the patient 101 on that day, e.g., feeling sedated or withdrawal. However, based on the Day 3+ method 500, it is recommended that the patient 101 not exceed 24 mg of buprenorphine-containing products in one day. The therapy application 160 and buprenorphine-containing product prescription and the program for the patient 101 may last any suitable number of days, such as seven days. Throughout each of the above methods 300, 400, 500, the patient 101 may input the time, date, and the dosage for each buprenorphine-containing product induction. The HCP 109 may provide any suitable input throughout the patient's 101 program, including modifying and tailoring the algorithms 300, 400, 500 for a specific patient 101, approving dosage amounts as suitable, etc. For any operation that requires waiting, the patient application 103 may “lock out” the patient 101 until the termination of the pre-determined time period, such that the patient 101 may not be able to access the patient application 103 until the termination of the pre-determined time period. In some implementations, the patient application 103 may display a countdown timer and may display an alert (e.g., visual and/or audial alert) upon termination of the pre-determined time period.

FIG. 6 illustrates a startup operation 600 for the patient application 103 executed by the therapy application 160 on the patient device 102. The patient application 103 is configured to starts at 602 upon the patient 101 accessing the patient application 103 by interacting with the patient device 102. The patient application 103 is configured to execute a login query at 604, prompting the patient 101 to input their login credentials, including the access code 104. The patient application 103 is configured to determine whether the inputted login credentials are valid at 606. If the patient application 103 determines that the inputted login credentials are invalid, the patient application 103 is configured to return to the login query at 604. If the patient application 103 determines that the inputted login credentials are valid, the patient application 103 is configured to determine which day of the patient's 101 program it currently is at 608.

If the patient application determines, at 608, that it is the first day of the patient's 101 program, the patient application 103 is configured to display initial information about buprenorphine-containing products at 610, as set forth above. The patient application 103 is then configured to execute a withdrawal symptom prompt at 612, as set forth above, which is configured to prompt the patient 101 to select or enter the withdrawal symptoms that the patient 101 is experiencing. The patient application 103 is then configured to determine, at 614, whether the patient 101 is experiencing, for example, at least five withdrawal symptoms. If the patient application 103 determines, at 614, that the patient 101 is experiencing less than five withdrawal symptoms, the patient application 103 is configured to return to the withdrawal symptom prompt 612. In some implementations, the patient application is configured to display a message to the patient 101 instructing the patient 101 to exit the patient application 103 and wait until the patient 101 is experiencing at least five withdrawal symptoms. If the patient application 103 determines, at 614, that the patient 101 is experiencing at least five withdrawal symptoms, the patient application 103 is configured to execute the Day 1 method or algorithm 300. At 608, if the patient application determines that it is the second day of the patient's 101 program, the patient application 103 is configured to execute the Day 2 method or algorithm 400. At 608, if the patient application determines that it is at least the third day of the patient's 101 program, the patient application 103 is configured to execute the Day 3+ method or algorithm 500.

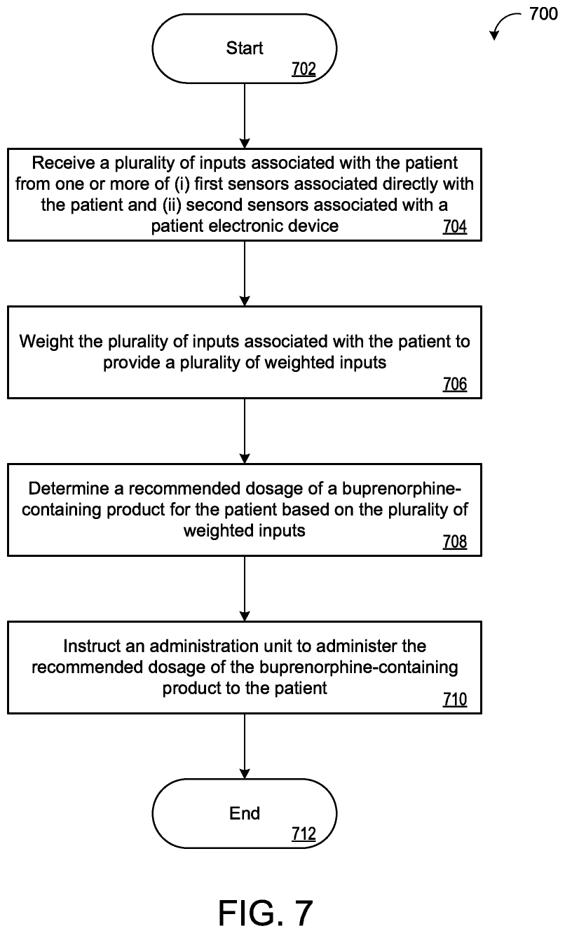

FIG. 7 is a flowchart illustrating a method 700 for implementing a prescription digital therapeutic configured to treat symptoms associated with opioid use disorder in a patient. The method 700 starts at 702 and includes receiving, at 704, a plurality of inputs associated with the patient from one or more of (i) first sensors associated directly with the patient and (ii) second sensors associated with a patient electronic device. The method 700 includes weighting, at 706, the plurality of inputs associated with the patient to provide a plurality of weighted inputs. The method 700 includes determining, at 708, a recommended dosage of a buprenorphine-containing product for the patient based on the plurality of weighted inputs. The method 700 includes instructing, at 710, an administration unit to administer the recommended dosage of the buprenorphine-containing product to the patient, and the method 700 ends at 712. It should be understood that the method 700 may include additional or fewer steps than those shown and described, and certain steps may be omitted or performed in any suitable order.

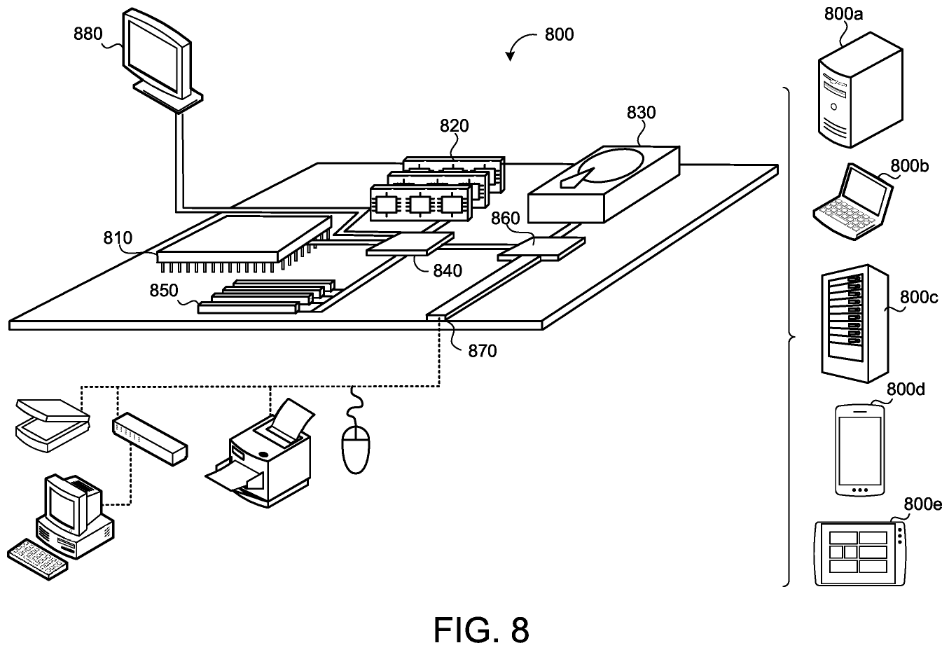

FIG. 8 is a schematic view of an example computing device 800 that may be used to implement the systems and methods described in this document. The computing device 800 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The computing device 800 includes a processor 810, memory 820, a storage device 830, a high-speed interface/controller 840 connecting to the memory 820 and high-speed expansion ports 850, and a low speed interface/controller 860 connecting to a low speed bus 870 and a storage device 830. Each of the components 810, 820, 830, 840, 850, and 860, are interconnected using various busses, and may be mounted on a common motherboard or in other manners as appropriate. The processor 810 can process instructions for execution within the computing device 800, including instructions stored in the memory 820 or on the storage device 830 to display graphical information for a graphical user interface (GUI) on an external input/output device, such as display 880 coupled to high speed interface 840. In other implementations, multiple processors and/or multiple buses may be used, as appropriate, along with multiple memories and types of memory. Also, multiple computing devices 800 may be connected, with each device providing portions of the necessary operations (e.g., as a server bank, a group of blade servers, or a multi-processor system).

The memory 820 stores information non-transitorily within the computing device 800. The memory 820 may be a computer-readable medium, a volatile memory unit(s), or non-volatile memory unit(s). The non-transitory memory 820 may be physical devices used to store programs (e.g., sequences of instructions) or data (e.g., program state information) on a temporary or permanent basis for use by the computing device 800. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random-access memory (DRAM), static random access memory (SRAM), phase change memory (PCM) as well as disks or tapes.

The storage device 830 is capable of providing mass storage for the computing device 800. In some implementations, the storage device 830 is a computer-readable medium. In various different implementations, the storage device 830 may be a floppy disk device, a hard disk device, an optical disk device, or a tape device, a flash memory or other similar solid-state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer- or machine-readable medium, such as the memory 820, the storage device 830, or memory on processor 810.

The high-speed controller 840 manages bandwidth-intensive operations for the computing device 800, while the low speed controller 860 manages lower bandwidth-intensive operations. Such allocation of duties is exemplary only. In some implementations, the high-speed controller 840 is coupled to the memory 820, the display 880 (e.g., through a graphics processor or accelerator), and to the high-speed expansion ports 850, which may accept various expansion cards (not shown). In some implementations, the low-speed controller 860 is coupled to the storage device 830 and a low-speed expansion port 890. The low-speed expansion port 890, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The computing device 800 may be implemented in a number of different forms, as shown in the figure. For example, it may be implemented as a standard server 800a or multiple times in a group of such servers 800a, as a laptop computer 800b, as part of a rack server system 800c, as a mobile device 800d (such as a smart phone), or as a tablet computer 800e.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random-access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that Seis used by the user; for example, by sending web pages to a web browser on a user's client device in response to requests received from the web browser.

A software application (i.e., a software resource) may refer to computer software that causes a computing device to perform a task. In some examples, a software application may be referred to as an “application,” an “app,” or a “program.” Example applications include, but are not limited to, system diagnostic applications, system management applications, system maintenance applications, word processing applications, spreadsheet applications, messaging applications, media streaming applications, social networking applications, and gaming applications.

The non-transitory memory may be physical devices used to store programs (e.g., sequences of instructions) or data (e.g., program state information) on a temporary or permanent basis for use by a computing device. The non-transitory memory may be volatile and/or non-volatile addressable semiconductor memory. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random-access memory (RAM), dynamic random-access memory (DRAM), static random-access memory (SRAM), phase change memory (PCM) as well as disks or tapes.

A number of implementations have been described. Nevertheless, it will be understood that various modifications may be made without departing from the spirit and scope of the disclosure. Accordingly, other implementations are within the scope of the following claims.

What is claimed is:

1. A system comprising:
data processing hardware; and
memory hardware in communication with the data processing hardware, the memory hardware storing instructions that when executed on the data processing hardware cause the data processing hardware to perform operations comprising:
executing a prescription digital therapeutic configured to treat symptoms associated with opioid use disorder in a patient, wherein executing the prescription digital therapeutic comprises:
receiving a plurality of inputs associated with the patient from one or more of (i) first sensors associated directly with the patient and (ii) second sensors associated with a patient electronic device, wherein the plurality of inputs represent a level of opioid withdrawal associated with the patient;
weighting the plurality of inputs associated with the patient to provide a plurality of weighted inputs;
determining a recommended dosage of a buprenorphine-containing product for the patient based on the plurality of weighted inputs; and
instructing an administration unit to administer the recommended dosage of the buprenorphine-containing product to the patient.

2. The system of claim 1, further comprising the administration unit, wherein the administration unit is configured to administer the recommended dosage of the buprenorphine-containing product to the patient based on the instructions.

3. The system of claim 2, wherein executing the prescription digital therapeutic further comprises administering the recommended dosage of the buprenorphine-containing product to the patient.

4. The system of claim 1, wherein the prescription digital therapeutic is configured to implement cognitive behavioral therapy to treat the symptoms associated with the opioid use disorder.

5. The system of claim 1, wherein the first sensors associated directly with the patient comprise one or more of: (i) a heart rate monitor, (ii) a blood pressure monitor, (iii) a sleep monitor, (iv) an electrodermal activity monitor, (v) a skin temperature sensor, and (vi) a sweat monitor.

6. The system of claim 1, wherein the second sensors associated with the patient electronic device comprise one or more of: (i) an accelerometer, (ii) a proximity sensor, and (iii) an activity monitor.

7. The system of claim 1, wherein weighting the plurality of inputs associated with the patient comprises assigning a first weight to a first input of the plurality of inputs and a second weight different than the first weight to a second input of the plurality of inputs.

8. The system of claim 7, wherein the first input of the plurality of inputs includes the first sensors associated directly with the patient and the second input of the plurality of inputs includes the second sensors associated with the patient electronic device.

9. The system of claim 8, wherein the first weight is greater than the second weight.

10. The system of claim 1, wherein the administration unit comprises one or more of: (i) a delivery pump, (ii) an injection unit, (iii) an implant, (iv) an oral absorption unit, (v) an inhaler, and (vi) a nasal injector.

11. A method comprising:
executing a prescription digital therapeutic configured to treat symptoms associated with opioid use disorder in a patient, wherein executing the prescription digital therapeutic comprises:
receiving, by data processing hardware, a plurality of inputs associated with the patient from one or more of (i) first sensors associated directly with the patient and (ii) second sensors associated with a patient electronic device, wherein the plurality of inputs represent a level of opioid withdrawal associated with the patient;
weighting, by the data processing hardware, the plurality of inputs associated with the patient to provide a plurality of weighted inputs;
determining, by the data processing hardware, a recommended dosage of a buprenorphine-containing product for the patient based on the plurality of weighted inputs; and
instructing, by the data processing hardware, an administration unit to administer the recommended dosage of the buprenorphine-containing product to the patient.

12. The method of claim 11, wherein the administration unit is configured to administer the recommended dosage of the buprenorphine-containing product to the patient based on the instructions.

13. The method of claim 12, wherein executing the prescription digital therapeutic includes administering, by the administration unit, the recommended dosage of the buprenorphine-containing product to the patient.

14. The method of claim 11, wherein the prescription digital therapeutic is configured to implement cognitive behavioral therapy to treat the symptoms associated with the opioid use disorder.

15. The method of claim 11, wherein the first sensors associated directly with the patient comprise one or more of: (i) a heart rate monitor, (ii) a blood pressure monitor, (iii) a sleep monitor, (iv) an electrodermal activity monitor, (v) a skin temperature sensor, and (vi) a sweat monitor.

16. The method of claim 11, wherein the second sensors associated with the patient electronic device comprise one or more of: (i) an accelerometer, (ii) a proximity sensor, and (iii) an activity monitor.

17. The method of claim 11, wherein weighting the plurality of inputs associated with the patient comprises assigning a first weight to a first input of the plurality of inputs and a second weight different than the first weight to a second input of the plurality of inputs.

18. The method of claim 17, wherein the first input of the plurality of inputs includes the first sensors associated directly with the patient and the second input of the plurality of inputs includes the second sensors associated with the patient electronic device.

19. The method of claim 18, wherein the first weight is greater than the second weight.

20. The method of claim 1, wherein the administration unit comprises one or more of: (i) a delivery pump, (ii) an injection unit, (iii) an implant, (iv) an oral absorption unit, (v) an inhaler, and (vi) a nasal injector.
